(19) United States
(12) Patent Application Publication
(10) Pub. No.: US 2004/0122067 A1
(43) Pub. Date: Jun. 24, 2004

(54) Treatment of chronic heart failure

(76) Inventor: Lin Zhao, Vernon Hills, IL (US)

Correspondence Address: STEVEN F. WEINSTOCK, ABBOTT LABORATORIES, 100 ABBOTT PARK ROAD, DEPT. 377/AP6A, ABBOTT PARK, IL, 60064-6008, US

(21) Appl. No.: 10/325,420

(22) Filed: Dec. 20, 2002

(51) Int. Cl. A61K 31/426; A61K 31/427

(52) U.S. Cl. 514/365

(57) ABSTRACT

Methods of treating chronic heart failure (CHF) by increasing the high energy phosphate concentrations available to heart muscle by administering a xanthine oxidase inhibitor to a patient having CHF are provided. As a result of such therapy, contractility of the heart is improved.

### TECHNICAL FIELD

The present invention relates to chronic heart failure (CHF) and, in particular, relates to increasing cardiac contractility by preventing high energy phosphate depletion in patients with CHF.

### BACKGROUND OF THE INVENTION

Chronic heart failure (CHF) is a common medical condition that affects approximately 2.0% of the population (4.8 million people) in the United States. It is the only major cardiovascular disorder that is increasing in incidence and prevalence due to the increased life span of the population. The disorder is annually responsible for over 11 million physician office visits and causes or contributes to over 3.5 million hospitalizations in people over 65 years old annually. Despite improvement in mortality in recent years, CHF remains a major source of cardiovascular healthcare expenditures.

CHF is a complex clinical syndrome that can be caused by a variety of disorders. For example, one common cause of CHF is ischemia which typically results in the reduction, or loss, of blood flow to a particular part of the heart due to blockage in an artery that would otherwise deliver blood to that part of the heart. Thrombolysis, angioplasty and by-pass surgery can return blood flow to the affected area of the heart and serve as successful treatments for ischemia. However, if such treatments are delayed, portions of the heart tissue experiencing the ischemic insult can be lost or die. This lost or dead tissue is referred to as infarcted. Ultimately, CHF results in a reduction of the hearts ability to pump blood, whether associated with ischemia and the resultant infarction of heart tissue, or any of the other causes. Patients having hearts compromised with CHF experience a reduced ability to tolerate strenuous activity and a reduction in life expectency.

Efforts have been made to find therapies to help CHF patients and in particular find therapies that help the hearts of these patients pump more efficiently. Much of such effort has been dedicated to finding calcium-sensitizers. Calcium binds to a special protein called troponin in cardiac muscle cells and is the trigger for a cascade of events that causes heart muscle to contract and therefore pump blood. While U.S. Pat. No. 6,191,136 has reported an increase in calcium sensitivity associated with xanthine oxidase inhibitor therapy in in-vitro studies, many clinical studies with other calcium sensitizers, however, have failed due to a tendency to increase mortality (see e.g. Massie, B. M., 15 years of heart-failure trials: what have we learned? Lancet, 1998. 352(Suppl 1): p. S129-33).

While reduced calcium sensitivity may be one factor causing cardiac muscle contractile dysfunction, there appear to be many other factors that acting in concert to damage the heart muscle. For example, patients with CHF have elevated circulating or tissue levels of norepinephrine, angiotensin II, aldosterone, endothelin and vasopressin that can act to adversely affect the structure and function of the heart; see e.g. Francis, G. S., et al., The neurohumoral axis in congestive heart failure. Ann Intern Med, 1984.101(3): p. 370-7. These neurohormonal factors not only increase the hemodynamic stresses on the ventricle by causing sodium retention and peripheral vasoconstriction, but may also exert direct toxic effects on the heart; see e.g. Mann, D. L., et al., Adrenergic effects on the biology of the adult mammalian cardiocyte. Circulation, 1992. 85(2): p. 790-804. CHF patients also have increased circulating and tissue levels of inflammatory cytokines (e.g., tumor necrosis factor α, TNFα) that can impair the viability and function of cardiac cells and the vascular system; see e.g. Sharma, R. et al., The role of inflammatory mediators in chronic heart failure: cytokines, nitric oxide, and endothelin-1. Int J Cardiol, 2000. 72(2): p. 175-86. There is also evidence that oxidative stress is increased systemically in patients with CHF. Plasma malondialdehyde (MDA), a marker of lipid peroxidation, is high in patients with ischemic and non-ischemic dilated cardiomyopathy, and seems to correlate directly with severity and chronicity of symptoms and inversely with cardiac function and exercise capacity; see Givertz, M. M. and W. S. Colucci, New targets for heart-failure therapy: endothelin, inflammatory cytokines, and oxidative stress. Lancet, 1998. 352 Suppl 1: p. S134-8. Oxygen-derived free radicals have been shown to directly cause myocardial contractile dysfunction, primarily by decreasing the excitation-contraction coupling and the responsiveness of myofilament to Ca²⁺; see e.g. Haque, R., H. Kan, and M. S. Finkel, Effects of cytokines and nitric oxide on myocardial E-C coupling. Basic Res Cardiol, 1998. 93 Suppl 1: p. 86-94. Free radicals may also contribute to the impaired vascular endothelium dependent relaxation (mediated by nitric oxide) in CHF patients by scavenging nitric oxide and forming peroxinitrate, which is one of the most potent cytotoxic free radicals; see Haddad, I. Y., et. al., Concurrent generation of nitric oxide and superoxide damages surfactant protein A. Am J Physiol, 1994. 267(3 Pt 1): p. L242-9. Moreover, overproduction of oxygen free radicals can potentiate cellular immune activation, and inflammatory cytokine production can also stimulate oxidative stress. Therefore, oxidative stress and cytokines may contribute synergistically to the progression of CHF, see Sharma, R. et al. Another contributor may be alternations in myocardial energy metabolism, e.g. decrease in energy reserve in the forms of creatine phosphate and ATP; see e.g. Vogt, A. M. and W. Kubler, Heart failure: is there an energy deficit contributing to contractile dysfunction? Basic Res Cardiol, 1998. 93(1): p. 1-10. Both animal experiments and clinical observations have indicated that there was energy depletion in failing heart muscle, and energy metabolism correlated with myocardial contractile dysfunction and the clinical severity of heart failure, see Neubauer, S., et al., 31P magnetic resonance spectroscopy in dilated cardiomyopathy and coronary artery disease. Altered cardiac high-energy phosphate metabolism in heart failure. Circulation, 1992. 86(6): p. 1810-8.

Links between hyperuricemia and cardiovascular diseases have long been recognized, although there has been controversy as to whether hyperuricemia is an independent risk factor for overall cardiovascular mortality and morbidity; see e.g. Fang, J. and M. H. Alderman, Serum uric acid and cardiovascular mortality the NHANES I epidemiologic follow-up study, 1971-1992. National Health and Nutrition Examination Survey. Jama, 2000. 283(18): p. 2404-10. With regard to CHF, many reported clinical studies which were designed to study the relationship between hyperuricemia and CHF have shown a high serum uric acid (UA) level to be a significant and strong marker of disease prognosis; see e.g. Yamada, T., et al., Serum uric acid level is an independent predictor of cardiovascular mortality in patients with chronic heart failure. J Mol Cell Cardiol, 2000. 32(11): p. A106. High serum UA levels have been associated with CHF. However, it is not known if high serum UA is simply an associated phenomenon or actually contributes to the occurrence and progression of CHF.

Current therapeutic interventions focus on utilizing neurohormonal antagonists (such as angiotensin converting enzyme (ACE) inhibitors, β-adrenergic receptor blockers) to slow down the progression of heart failure together with diuretics for treatment of fluid retention (Am J Cardiol, 1999. 83(2A): p. 1A-38A). Long-term treatment with these drugs can improve clinical status and decrease the risk of major cardiac events. However, they often do not produce immediate symptomatic benefits.

There is much room left for improvement in drug treatment. None of the currently approved CHF treatments offers both long-term efficacy (inhibition or prevention of disease progression) as well as immediate symptom relief (increase cardiac output by positive inotropic activity). Furthermore, there are no approved CHF treatments which target the increased inflammatory cytokines, oxidative stress and energy depletion observed in patients (Lancet, 1998.352 Suppl 1: p. SI34-8).

Accordingly, there is a need to treat multiple mechanisms of decreased contractility in CHF patients to thereby alleviate symptoms, improve quality of life, to avoid significant increase in mortality, and to decrease the likelihood of disease progression, thereby decreasing the risk of death and the need for hospitalization along with its attendant costs.

### SUMMARY OF THE INVENTION

Methods for increasing cardiac contractility in a CHF patient by increasing the high energy phosphate molecule concentration in heart muscle of the patient, methods of increasing high energy phosphate concentrations in heart muscle of a patient having CHF, and methods of treating CHF are provided herein. According to any of the methods, the methods comprise administering to a CHF patient, or a patient in need of such therapy, a therapeutically effective amount of a xanthine oxidase inhibitor compound.

### DETAILED DESCRIPTION OF THE INVENTION

Biochemical reactions that occur in cells often times require energy. A prime source of energy used by cells is adenine triphosphate (ATP). In the case of heart muscle cells, ATP and phosphocreatine (PCr) (which is synthesized by consuming ATP) are the primary sources of energy to maintain contraction. The concentrations of these “high energy phosphate molecules” are reduced in patients suffering from CHF and therefore the ability of their hearts to contract is compromised due to a lack of energy. It has been discovered that xanthine oxidase inhibitor compounds variously referred to herein as (“xanthine oxidase inhibitors”) contribute to ATP conservation in patients experiencing CHF. Administering xanthine oxidase inhibitor compounds to patients suffering from CHF increases the ATP concentration in such patients' heart muscle above the concentration that their heart muscle would otherwise have in the absence of a xanthine oxidase inhibitor compound. Hence, providing a patient suffering from CHF with xanthine oxidase inhibitors increases ATP concentration (and consequently PCr) in the cells of heart muscle and thereby increases the contractility of the heart.

As mentioned above, the presence and activity of xanthine oxidase, as well as the products of its activity, in patients suffering from CHF is elevated. While it is not completely understood, and not wishing to be bound by theory, it is believed that xanthine oxidase inhibitors increase ATP concentration by preventing the irreversible breakdown of ATP caused by the activity of xanthine oxidase. As a result, more ATP and therefore more PCr, is available to enable heart muscle to contract more efficiently.

Specifically, the metabolic pathway for ATP degradation is as follows:

ATP⇄ADP⇄AMP⇄Adenosine⇄Inosine⇄Hypoxanthine→Xanthine→Uric Acid

As seen by the pathway presented above, ATP is reversibly broken down to various metabolites up to the point where hypoxanthine is converted to xanthine. Once this reaction occurs, there is no “salvage” pathway to convert xanthine, or its downstream degradation product uric acid, back to ATP. The enzyme responsible for converting hypoxanthine to xanthine is xanthine oxidase. Hence, inhibiting xanthine oxidase would prevent breakdown of hypoxanthine and allow it to be converted back to ATP via the salvage pathway. As alluded to above, the presence of uric acid in CHF patients is elevated, therefore indicating the activity of xanthine oxidase in such patients. Hence, providing an inhibitor of xanthine oxidase would allow ATP to be generated from hypoxanthine and, in the muscle of the heart, provide additional energy to enable it to contract more effectively. Accordingly, administering xanthine oxidase inhibitor compounds to CHF patients increases the ATP concentration in the heart muscle by preventing the breakdown of ATP to by-products that cannot be converted back to molecules that provide cells with energy used to, for example, contract heart muscle.

Xanthine oxidase inhibitor compounds that can be used according to the present invention include any pharmaceutically acceptable compound having the ability to decrease the activity of xanthine oxidase. As used herein, the term “pharmaceutically acceptable” as used herein includes moieties or compounds that are, within the scope of sound medical judgment, suitable for use in contact with the tissues of humans and lower animals without undue toxicity, irritation, allergic response, and the like, and are commensurate with a reasonable benefit/risk ratio. Oxypurinol and allopurinol are well known examples of pharmaceutically acceptable xanthine inhibitor compounds. Additionally, xanthine oxidase inhibitor compounds described in U.S. Pat. Nos. 2,868,803; 3,474,098; 3,624,205;3,890,313; 3,892,738; 3,892,858; 3,907,799; 3,920,652; 4,021,556; 4,024,253; 4,058,614; 4,179,512; 4,241,064; 4,281,005; 4,346,094; 4,495,195; 5,212,201; 5,272,151; and 5,674,887 are also suitable. Methods for synthesizing such compounds are also disclosed in the above patents. As is well known in the art, other suitable xanthine oxidase inhibitor compounds can be found using xanthine oxidase and xanthine in assays to determine if such candidate compounds inhibit conversion of hypoxanthine into xanthine or uric acid.

Xanthine oxidase inhibitor compounds having the following formula (I) are particularly preferred:

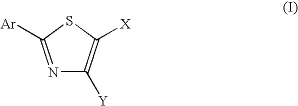

wherein Ar is an unsubstituted or substituted furyl group; or a group represented by the following formula (II):

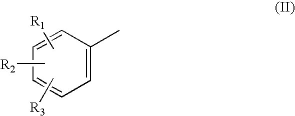

wherein R₁, R₂, and R₃ are hydrogen, a halogen atom, or a nitro, cyano or formyl group; or a group of OR₄, S(O)ₙR₅ and NR₆R₇ (wherein n is an integer of from 1 to 2, R₄, R₅, and R₆, each may independently represent an unsubstituted or substituted C₁₋₁₀ alkyl, aryl, aralkyl, alkylcarbonyl, arylcarbonyl or aralkylcarbonyl group, R₇ represents a hydrogen atom, or an unsubstituted or substituted C₁₋₁₀ alkyl, aryl, aralkyl, alkylcarbonyl, arylcarbonyl or aralkylcarbonyl group; or R₆ and R₇, taken together with the nitrogen atom bonded thereto, represent atoms forming an unsubstituted or substituted 5- or 7-membered heterocyclic ring), or a group of COR₈ wherein R₈ represents an unsubstituted or substituted C₁₋₁₀ alkyl, aryl or aralkyl group; a hydroxyl group; an unsubstituted or substituted C₁₋₁₀ alkoxy, aryloxy or aralkyloxy group; an amino group; or an unsubstituted or substituted C₁₋₁₀ alkyl (mono- or di-substituted, independently) amino, aryl (mono- or di-substituted, independently) amino or aralkyl (mono- or di-substituted, independently) amino group, or a 5- to 7-membered cyclic amino group, and at least one of R₁, R₂ or R₃ is other than hydrogen;

X is a hydrogen atom, or a C₁₋₁₄ alkyl, carboxyl, C₁₋₅ alkoxycarbonyl, carbamoyl, C₁₋₄ alkyl (mono- or di-substituted) aminocarbonyl group or COOR₉, where R₉ is a C₁₋₄ alkyl group; and

Y represents a hydrogen atom or a C₁₋₄ alkyl, carboxyl, C₁₋₅ alkoxycarbonyl, carbamoyl or C₁₋₄ alkyl (mono- or di-substituted) aminocarbonyl group, with the proviso that when at least one group of R₁, R₂ and R₃ represents a halogen atom, or an alkoxy, alkylamino or nitro group, at least one group of the two other groups represents a group other than a hydrogen atom; when at least one group of R₁, R₂ and R₃ is a halogen atom and another group is a hydrogen atom, a remaining group is a group other than a halogen atom, or an alkoxy, alkylamino or acylamino group, with the additional proviso that when any one of R₁, R₂ or R₃ is OR₄, one of the remaining groups cannot represent hydrogen while the other group represents OR₄, or the remaining two groups cannot both represent OR₄ at the same time, nor do all of R₁-R₃ represent halogen; with the further proviso that both X and Y do not represent carboxyl, C₁₋₅ alkoxycarbonyl, carbamoyl or C₁₋₄ alkyl (a mono- or di-substituted) aminocarbonyl group at the same time. Such compounds, as well as methods for synthesizing them are described in U.S. Pat. No. 5,614,520.

Compounds used in accordance with present invention can be provided in the form of pharmaceutically acceptable salts derived from inorganic or organic acids. Pharmaceutically acceptable salts are well-known in the art. For example, S. M. Berge et al. describe pharmaceutically acceptable salts in detail in J. Pharmaceutical Sciences, 1977, 66: 1 et seq. The salts can be prepared in situ during the final isolation and purification of the compounds or separately by reacting a free base function with a suitable organic acid. Representative acid addition salts include, but are not limited to acetate, adipate, alginate, citrate, aspartate, benzoate, benzenesulfonate, bisulfate, butyrate, camphorate, camphor sulfonate, digluconate, glycerophosphate, hemisulfate, heptanoate, hexanoate, fumarate, hydrochloride, hydrobromide, hydroiodide, 2-hydroxyethansulfonate (isothionate), lactate, maleate, methane sulfonate, nicotinate, 2-naphthalene sulfonate, oxalate, palmitoate, pectinate, persulfate, 3-phenylpropionate, picrate, pivalate, propionate, succinate, tartrate, thiocyanate, phosphate, glutamate, bicarbonate, p-toluenesulfonate and undecanoate. Also, basic nitrogen-containing groups can be quaternized with such agents as lower alkyl halides such as methyl, ethyl, propyl, and butyl chlorides, bromides and iodides; dialkyl sulfates like dimethyl, diethyl, dibutyl and diamyl sulfates; long chain halides such as decyl, lauryl, myristyl and stearyl chlorides, bromides and iodides; arylalkyl halides like benzyl and phenethyl bromides and others. Water or oil-soluble or dispersible products are thereby obtained. Examples of acids which can be employed to form pharmaceutically acceptable acid addition salts include such inorganic acids as hydrochloric acid, hydrobromic acid, sulphuric acid and phosphoric acid and such organic acids as oxalic acid, maleic acid, succinic acid and citric acid.

Basic addition salts can be prepared in situ during the final isolation and purification of compounds by reacting a carboxylic acid-containing moiety with a suitable base such as the hydroxide, carbonate or bicarbonate of a pharmaceutically acceptable metal cation or with ammonia or an organic primary, secondary or tertiary amine. Pharmaceutically acceptable salts include, but are not limited to, cations based on alkali metals or alkaline earth metals such as lithium, sodium, potassium, calcium, magnesium and aluminum salts and the like and nontoxic quaternary ammonia and amine cations including ammonium, tetramethylammonium, tetraethylammonium, methylammonium, dimethylammonium, trimethylammonium, triethylammonium, diethylammonium, and ethylammonium among others. Other representative organic amines useful for the formation of base addition salts include ethylenediamine, ethanolamine, diethanolamine, piperidine, piperazine and the like.

Xanthine oxidase inhibitor compounds, or salts thereof, may be formulated in a variety of ways that is largely a matter of choice depending upon the delivery route desired. For example, Solid dosage forms for oral administration include capsules, tablets, pills, powders and granules. In such solid dosage forms, the xanthine oxidase inhibitor compound may be mixed with at least one inert, pharmaceutically acceptable excipient or carrier, such as sodium citrate or dicalcium phosphate and/or a) fillers or extenders such as starches, lactose, sucrose, glucose, mannitol and silicic acid; b) binders such as carboxymethylcellulose, alginates, gelatin, polyvinylpyrrolidone, sucrose and acacia; c) humectants such as glycerol; d) disintegrating agents such as agar-agar, calcium carbonate, potato or tapioca starch, alginic acid, certain silicates and sodium carbonate; e) solution retarding agents such as paraffin; f) absorption accelerators such as quaternary ammonium compounds; g) wetting agents such as cetyl alcohol and glycerol monostearate; h) absorbents such as kaolin and bentonite clay and i) lubricants such as talc, calcium stearate, magnesium stearate, solid polyethylene glycols, sodium lauryl sulfate and mixtures thereof.

Solid compositions of a similar type may also be employed as fillers in soft and hard-filled gelatin capsules using such excipients as lactose or milk sugar as well as high molecular weight polyethylene glycols and the like.

The solid dosage forms of tablets, capsules, pills and granules can be prepared with coatings and shells such as enteric coatings and other coatings well-known in the pharmaceutical formulating art. They may optionally contain opacifying agents and may also be of a composition such that they release the active ingredient(s) only, or preferentially, in a certain part of the intestinal tract, optionally, in a delayed manner. Examples of embedding compositions which can be used include polymeric substances and waxes.

Liquid dosage forms for oral administration include pharmaceutically acceptable emulsions, solutions, suspensions, syrups and elixirs. In addition to the xanthine oxidase inhibitor compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl henzoate, propylene glycol, 1,3-butylene glycol, dimethyl formamide, oils (in particular, cottonseed, groundnut, corn, germ, olive, castor and sesame oils), glycerol, tetrahydrofurfuryl alcohol, polyethylene glycols and fatty acid esters of sorbitan and mixtures thereof.

The compositions can also be delivered through a catheter for local delivery at a target site, via an intracoronary stent (a tubular device composed of a fine wire mesh), or via a biodegragable polymer.

Compositions suitable for parenteral injection may comprise physiologically acceptable, sterile aqueous or nonaqueous solutions, dispersions, suspensions or emulsions and sterile powders for reconstitution into sterile injectable solutions or dispersions. Examples of suitable aqueous and nonaqueous carriers, diluents, solvents or vehicles include water, ethanol, polyols (propyleneglycol, polyethyleneglycol, glycerol, and the like), vegetable oils (such as olive oil), injectable organic esters such as ethyl oleate, and suitable mixtures thereof.

These compositions can also contain adjuvants such as preserving, wetting, emulsifying, and dispensing agents. Prevention of the action of microorganisms can be ensured by various antibacterial and antifungal agents, for example, parabens, chlorobutanol, phenol, sorbic acid, and the like. It may also be desirable to include isotonic agents, for example sugars, sodium chloride and the like. Prolonged absorption of the injectable pharmaceutical form can be brought about by the use of agents delaying absorption, for example, aluminum monostearate and gelatin.

Suspensions, in addition to the active compounds, may contain suspending agents, as for example, ethoxylated isostearyl alcohols, polyoxyethylene sorbitol and sorbitan esters, microcrystalline cellulose, aluminum metahydroxide, bentonite, agar-agar and tragacanth, or mixtures of these substances, and the like.

Proper fluidity can be maintained, for example, by the use of coating materials such as lecithin, by the maintenance of the required particle size in the case of dispersions and by the use of surfactants.

In some cases, in order to prolong the effect of the drug, it is desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle. Injectable depot forms are made by forming microeneapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissues.

The injectable formulations can be sterilized, for example, by filtration through a bacterial-retaining filter or by incorporating sterilizing agents in the form of sterile solid compositions which can be dissolved or dispersed in sterile water or other sterile injectable medium just prior to use.

Dosage forms for topical administration of a compound of this invention include powders, sprays, ointments and inhalants. The active compound is mixed under sterile conditions with a pharmaceutically acceptable carrier and any needed preservatives, buffers or propellants which can be required. Opthalmic formulations, eye ointments, powders and solutions are also contemplated as being within the scope of this invention.

It will be understood that formulations used in accordance with the present invention generally will comprise a therapeutically effective amount of one or more xanthine oxidase inhibitor compounds. The phrase “therapeutically effective amount” as used herein means a sufficient amount of, for example, the composition, xanthine oxidase inhibitor compound, or formulation necessary to treat the desired disorder, at a reasonable benefit/risk ratio applicable to any medical treatment. As with other pharmaceuticals, it will be understood that the total daily usage of a pharmaceutical composition of the invention will be decided by a patient's attending physician within the scope of sound medical judgment. The specific therapeutically effective dose level for any particular patient will depend upon a variety of factors including the disorder being treated and the severity of the disorder; activity of the specific compound employed; the specific composition employed; the age, body weight, general health, sex and diet of the patient; the time administration, route of administration, and rate of excretion of the specific compound employed; the duration of the treatment; drugs used in combination or coincidental with the specific compound employed; and other factors known to those of ordinary skill in the medical arts. For example, it is well within the skill of the art to start doses of the compound at levels lower than required to achieve the desired therapeutic effect and to gradually increase the dosage until the desired effect is achieved.

Formulations of the invention are administered and dosed in accordance with sound medical practice, taking into account the clinical condition of the individual patient, the site and method of administration, scheduling of administration, and other factors known to medical practitioners.

Therapeutically effective amounts for purposes herein thus can readily be determined by such considerations as are known in the art. The daily pharmaceutically effective amount of the compounds administered to a patient in single or divided doses range from about 0.1 to about 200 mg/kg body weight, preferably from about 0.25 to about 200 mg/kg body weight. Most typically, a typical daily dose is between 25 mg/day and 400 mg/day, preferably between 50 mg/day and 300 mg/day.

Generally, the methods of the invention comprise administration of a therapeutically effective amount of a xanthine oxidase inhibitor compound to a patient having CHF to thereby increase the high energy phosphate concentrations in the heart muscle and consequently increasing cardiac contractility or allowing the heart to contract more effectively than it would in the absence of a xanthine oxidase inhibitor compound. Hence a method of treating CHF is provided that increases cardiac contractility by increasing the concentrations of high energy phosphate available to the heart muscle.

CHF patients are preferably those who have survived cardiac insult such as ischemia, ischemia-reperfusion injury, or any of the other causes of cardiogenic shock. Hence, xanthine oxidase inhibitors of the present invention can immediately be administered to CHF patients or, for example, after several hours, days, weeks or months after the event causing the cardiogenic shock. While short term regimens are contemplated, since the hearts of CHF patients are chronically compromised in their ability to contract, relatively regular and long term administration of xanthine oxidase inhibitors to achieve the above results are also contemplated. Hence, xanthine oxidase inhibitors can be administered regularly after cardiogenic shock on a short term basis such as for one or more days, weeks, or months; or xanthine oxidase inhibitors can be administered for one or more years to achieve the beneficial effects described above.

It should be understood that the invention is not invalidated or limited in any way should a particular theory or proposed mechanism of action prove to be wrong in the future.

While the invention is described above in connection with preferred or illustrative embodiments, these embodiments are not intended to be exhaustive or limiting of the invention. Rather, the invention is intended to cover all alternatives, modifications and equivalents included within its spirit and scope of the invention, as defined by the appended claims.

What is claimed:

1. A method for increasing cardiac contractility in a CHF patient by increasing the high energy phosphate molecule concentration in heart muscle of the patient comprising administering a therapeutically effective amount of a xanthine oxidase inhibitor compound to the patient.

2. A method of increasing high energy phosphate concentrations in heart muscle of a patient having CHF comprising administering a xanthine oxidase inhibitor compound to the patient.

3. A method of treating C₁₄F comprising administering to a CHF patient a therapeutically effective amount of a xanthine oxidase inhibitor compound.

4. The method of claim 1 wherein the xanthine oxidase inhibitor compound has the formula (I) or a pharmaceutically acceptable salt thereof: 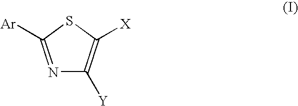
wherein Ar is an unsubstituted or substituted furyl group; or a group represented by the following formula (II): 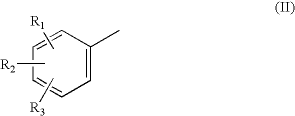
wherein R₁, R₂, and R₃ are hydrogen, a halogen atom, or a nitro, cyano or formyl group; or a group of OR₄, S(O)ₙR₅ and NR₆R₇ (wherein n is an integer of from 1 to 2, R₄, R₅, and R₆, each may independently represent an unsubstituted or substituted C₁₋₁₀ alkyl, aryl, aralkyl, alkylcarbonyl, arylcarbonyl or aralkylcarbonyl group, R₇ represents a hydrogen atom, or an unsubstituted or substituted C₁₋₁₀ alkyl, aryl, aralkyl, alkylcarbonyl, arylcarbonyl or aralkylcarbonyl group; or R₆ and R₇, taken together with the nitrogen atom bonded thereto, represent atoms forming an unsubstituted or substituted 5- or 7-membered heterocyclic ring), or a group of COR₈ wherein R₈ represents an unsubstituted or substituted C₁₋₁₀ alkyl, aryl or aralkyl group; a hydroxyl group; an unsubstituted or substituted C₁₋₁₀ alkoxy, aryloxy or aralkyloxy group; an amino group; or an unsubstituted or substituted C₁₋₁₀ alkyl (mono- or di-substituted, independently) amino, aryl (mono- or di-substituted, independently) amino or aralkyl (mono- or di-substituted, independently) amino group, or a 5- to 7-membered cyclic amino group, and at least one of R₁, R₂ or R₃ is other than hydrogen;
X is a hydrogen atom, or a C₁₋₁₄ alkyl, carboxyl, C₁₋₅ alkoxycarbonyl, carbamoyl, C₁₋₄ alkyl (mono- or di-substituted) aminocarbonyl group or COOR₉, where R₉ is a C₁₋₄ alkyl group; and
Y represents a hydrogen atom or a C₁₋₄ alkyl, carboxyl, C₁₋₅ alkoxycarbonyl, carbamoyl or C₁₋₄ alkyl (mono- or di-substituted) aminocarbonyl group, with the proviso that when at least one group of R₁, R₂ and R₃ represents a halogen atom, or an alkoxy, alkylamino or nitro group, at least one group of the two other groups represents a group other than a hydrogen atom; when at least one group of R₁, R₂ and R₃ is a halogen atom and another group is a hydrogen atom, a remaining group is a group other than a halogen atom, or an alkoxy, alkylamino or acylamino group, with the additional proviso that when any one of R₁, R₂ or R₃ is OR₄, one of the remaining groups cannot represent hydrogen while the other group represents OR₄, or the remaining two groups cannot both represent OR₄ at the same time, nor do all of R₁-R₃ represent halogen; with the further proviso that both X and Y do not represent carboxyl, C₁₋₅ alkoxycarbonyl, carbamoyl or C₁₋₄ alkyl (a mono- or di-substituted) aminocarbonyl group at the same time.
